(19) United States
(12) Patent Application Publication
(10) Pub. No.: US 2009/0074679 A1
(43) Pub. Date: Mar. 19, 2009

(54) TOOTH WHITENING METHODS AND APPARATUSES

(76) Inventor: Harvey N. Silverman, Pepper Pike, OH (US)

Correspondence Address: Fay Sharpe LLP, 1228 Euclid Avenue, 5th Floor, The Halle Building, Cleveland, OH, 44115-1843, US

(21) Appl. No.: 11/854,714

(22) Filed: Sep. 13, 2007

(51) Int. Cl. A61K 8/22; A61C 17/00; A61Q 11/00

(52) U.S. Cl. 424/53; 424/49; 433/217.1

(57) ABSTRACT

Some disclosed tooth whitening methods comprise: painting a tooth whitening solution or gel including a bleaching agent onto teeth; and subsequent to the painting, disposing a conforming protective cover over the teeth. In some embodiments, the painting and disposing are performed under the non-interventional guidance of a session guide. In some tooth whitening article of manufacture embodiments, a tooth whitening solution or gel includes a bleaching agent, the tooth whitening solution or gel being configured for paint-on application onto teeth, and conforming protective cover is configured to be conformally disposed over the teeth after painting with the tooth whitening solution or gel. In some embodiments a tooth whitening solution or gel includes a bleaching agent at hydrogen peroxide-equivalent concentration of greater than or about 10 percent and does not include a reactive thickening agent.

### BACKGROUND

The following relates to the dental arts. It finds particular application in over-the-counter tooth whitening approaches. However, the following will find application in tooth whitening generally, including tooth whitening procedures performed in the dental office or similar settings.

White teeth are widely regarded as cosmetically attractive and desirable, both in the United States and in numerous other countries. However, the whiteness of teeth can be adversely affected over time by oral activities including eating, drinking, smoking, and so forth. Accordingly, there is substantial interest in development of safe and effective tooth whitening methods and apparatuses.

Some tooth whitening formulations employ a bleaching agent such as hydrogen peroxide. In the oral environment, hydrogen peroxide decomposes to produce oxygen free radicals that react with tooth discoloring chromophoric substances so as to enhance tooth whiteness. It is known that the whitening effect generally increases with increasing concentration of the hydrogen peroxide or other bleaching agent, and generally increases with longer application times.

Problematically, hydrogen peroxide concentrations above about 10% tend to lead to substantial irritation of the gums, lips, and other soft oral tissue. Excessive exposure of the tooth enamel to the bleaching agent can also lead to a phenomenon called “overwhitening” which manifests as an apparent bluish discoloration of the teeth that is generally deemed undesirable. Still further, in some subjects tooth sensitivity is induced by the tooth whitening process, sometimes to the point of motivating toward the use of analgesics for pain control.

Accordingly, tooth whitening procedures employing high bleaching agent concentrations are generally performed in the dental office. Skilled dental professionals use lip retractors to space the lips apart from the teeth. A protective coating is applied in the vicinity of the gum line to protect the gum tissue. Once these protective measures have been taken, the dental professional applies a tooth whitening solution including a relatively high concentration of bleaching agent to the teeth. The whitening solution may be applied using a paint-on approach in which the whitening solution is applied to the teeth using a brush, swap, nozzle, or other instrument. In another approach, a tray containing the whitening solution is placed over the teeth. The tray is fitted to the specific patient's dental configuration by making a tray custom-formed to match the patient's dental pattern.

These approaches have certain disadvantages. As relatively invasive dental procedures, they are required by law in the United States and some other countries to be performed in the dental office by licensed or otherwise suitably qualified dentists. Even absent a legal mandate, the procedures are generally best performed by dental professionals, and are not suitable for self-application. The processes are uncomfortable and relatively expensive for the patient. Even with the assistance of skilled dental personnel, there remains a substantial possibility of occurrence of overwhitening and/or induced tooth sensitivity. The use of a tray, in particular, is susceptible to overwhitening since the dosage of whitening agent is difficult to precisely control.

As an alternative, numerous over-the-counter tooth whitening procedures have been developed, which are designed to be performed by a consumer at home without the intervention of a professional dentist. Generally, these over-the-counter procedures fall into three categories—paint-on whitening solutions, and whitening strips or trays impregnated with a whitening solution; and whitening trays such as so-called “boil-and-bite” trays that contain a whitening solution. Because the consumer does not have access to the lip retractors, gum sealants, and other protective accessories that are employed at the dental office, over-the-counter whitening solutions typically have substantially reduced concentration of bleaching agent as compared with those used in dental offices, so as to reduce or avoid irritation or damage to the gums, lips, or other soft oral tissues.

To compensate for the reduced concentration, the time of application is lengthened in the case of some over-the-counter whiteners. In some approaches, a reactive thickening agent such as Carbopol® is used, which tends to bind to the hydrogen peroxide or other bleaching agent so as to buffer or slow down the bleaching process. The theory is that by such buffering, the bleaching agent can be applied for a longer period of time, so as to provide more effective whitening. As another approach, a bleaching agent with lower reactivity, such as percarbonate peroxide, urea peroxide, or carbamide peroxide, is used in place of hydrogen peroxide. These other peroxide-based bleaching agents typically have an effective concentration of about ⅓ the equivalent concentration of hydrogen peroxide, due to their lower reactivity. For example, a 15% percarbonate peroxide-based whitening solution is effectively equivalent to about a 5% hydrogen peroxide-based whitening solution. Again, the theory is that a lower reactivity bleaching agent can be applied for a longer period of time without producing soft tissue irritation or other adverse effects, so as to provide more effective whitening. The effective concentration, that is, the hydrogen peroxide-equivalent concentration, is typically kept at or below about 10%, and more typically close to about 5-6%. For example, some existing over-the-counter tooth whitening products have been reported to use 10-18% carbamide peroxide, while other existing over-the-counter tooth whitening products have been reported to use about 5-6% hydrogen peroxide. Whitening products with more than 10% hydrogen peroxide-equivalent concentration are generally not sold as over-the-counter products.

Existing over-the-counter whitening approaches have certain disadvantages. The low bleaching agent concentration leads to concomitantly long application times, typically on the order of a half-hour to an hour or longer, which is inconvenient for the consumer. In the case of paint-on whiteners, the long application time enhances the likelihood that the lips, tongue, or other soft oral tissues will inadvertently contact and remove the whitening agent. In the case of whitening strips, longer wear time can result in induced tooth sensitivity in spite of relatively low concentrations of bleaching agent. Some consumers also report excessive salivation which can lead to drooling.

Another disadvantage of both paint-on whiteners and whitening strips is that they typically are removed at a bathroom sink—the paint-on whitener being removed by brushing it off with a toothbrush, and the whitening strip being removed and thrown in the trash. This can be inconvenient. Whitening strips that can be swallowed have been developed, but the low concentration of hydrogen peroxide in combination with the short wear time produces less than desirable results.

In addition, over-the-counter whitening products have the disadvantage of being performed in a wholly unassisted manner. While it is advantageous from a cost and convenience standpoint to avoid a visit to the dentist, it is also desirable to get initial guidance as to how to perform the tooth whitening process, and to get skilled feedback as to whether the treatment is showing positive effect. Because the whitening improvement is incremental with successive whitening sessions, it is sometimes difficult for the consumer to determine if a single over-the-counter tooth whitening session produced a measurable amount of tooth whitening. Because of the long application times involved, it is inconvenient for the consumer to seek and obtain guidance from the point-of-purchase in using the product since such assistance would entail the consumer remaining at the point-of-purchase for up to several hours while the whitening session proceeds, or scheduling a return visit to the point-of-purchase, in order to receive feedback on its success or failure. Moreover, store clerks, check-out personnel or other retail employees are typically not trained in the art and science associated with tooth whitening.

### BRIEF SUMMARY

In an illustrative method disclosed herein as an illustrative example, a tooth whitening method is disclosed, comprising: painting a tooth whitening solution or gel including a bleaching agent onto teeth; and subsequent to the painting, disposing a conforming protective cover over the teeth.

In an illustrative article of manufacture disclosed herein as an illustrative example, a tooth whitening article of manufacture is disclosed, comprising: a tooth whitening solution or get including a bleaching agent, the tooth whitening solution or get configured for paint-on application onto teeth; and a conforming protective cover configured to be conformally disposed over the teeth after painting with the tooth whitening solution or gel.

In an illustrative article of manufacture disclosed herein as an illustrative example, a tooth whitening solution or gel comprises a peroxide-based bleaching agent at a hydrogen peroxide-equivalent concentration of greater than or about 10 percent, wherein the tooth whitening solution or gel does not include a reactive thickening agent and includes an accelerant that accelerates decomposition of the peroxide-based bleaching agent.

### BRIEF DESCRIPTION OF THE DRAWINGS

FIG. 1 diagrammatically illustrates a tooth whitening article of manufacture.

FIG. 2 diagrammatically shows a paint-on process operation of a tooth whitening process performed using the tooth whitening article of manufacture of FIG. 1.

FIG. 3 diagrammatically shows a protective strip or microtray application process operation of a tooth whitening process performed using the tooth whitening article of manufacture of FIG. 1.

FIG. 4 diagrammatically shows a guided training process for training a consumer on using the tooth whitening article of manufacture of FIG. 1.

### DETAILED DESCRIPTION

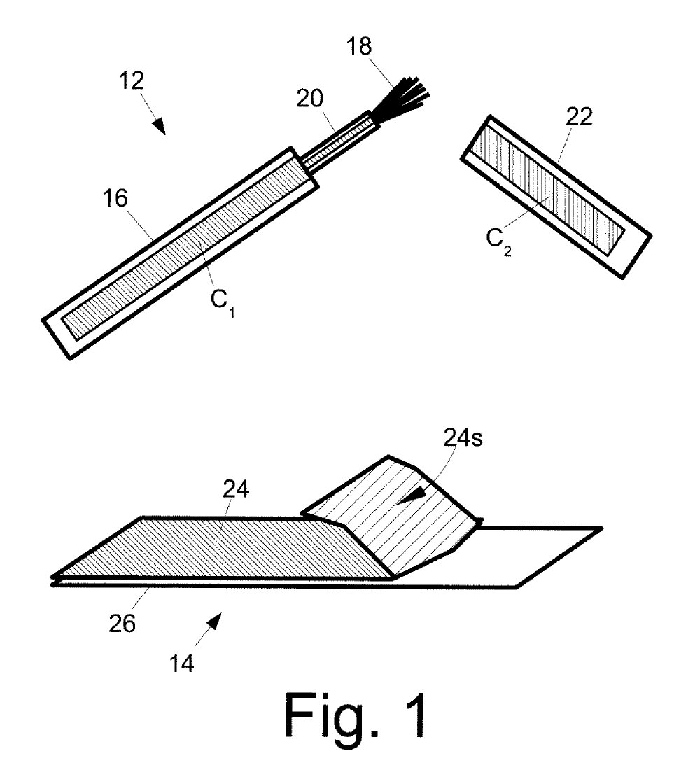

With reference to FIG. 1, a tooth whitening article of manufacture includes an applicator 12 for applying a tooth whitening solution to teeth, and a protective wrap or microtray assembly 14 configured to wrap the teeth after the tooth whitening solution is applied. The illustrated applicator 12 is a pen-type applicator including a handle 16 and an application element such as an illustrated brush 18, a cotton swab tip, a sponge tip, a nozzle, a sealed ampoule, or so forth. In some embodiments, the handle 16 is substantially hollow and a tooth whitening solution or tooth whitening solution component C₁ (not visible in the assembled applicator 12, and hence shown in shaded phantom in FIG. 1) is disposed in the handle 16 and is driven to the brush 18 by a manual pumping action built into the handle 16. For example, by twisting or pushing a connecting handle portion 20 respective to a bulk of the handle 16, or in some embodiments by squeezing the handle 20, tooth whitening solution or solution component C₁ can be pumped or driven toward the brush 18. In some embodiments, a passive drawing mechanism such as wicking or capillary action is provided to draw the tooth whitening solution or solution component C₁ to the brush 18 without active action by the consumer. In some embodiments, the applicator includes a container such as an ampoule containing the whitening solution or solution component C₁ and further having a cap with a built-in applicator such as the illustrated brush 18, a sponge tip, swab tip, or so forth that can be dipped into the opening presented by the removed cap to access the whitening solution or solution component C₁.

In other embodiments, a separate container, such as an optional illustrated tubular container 22, contains a tooth whitening solution or tooth whitening solution component C₂ (not visible in the assembled applicator 12, and hence shown in shaded phantom in FIG. 1) which is applied to the brush 18 by dipping the brush 18 into the container 22. Instead of being a second paint-on component, the second component C₂ can be embodied as a film disposed on a protective cover, or a solution impregnated in a protective cover, and the protective cover is applied over the teeth after the paint-on process is complete.

In some embodiments, the tooth whitening solution is a one-component solution, for example including only the tooth whitening solution C₁ or only the tooth whitening solution C₂. In some embodiments, the tooth whitening solution is a two-component solution, for example with first component C₁ stored in a hollow portion of the handle 16 as described, and second component C₂ stored in the separate tubular container 22. The two components C₁, C₂ are combined by drawing or pumping the solution component C₁ in the handle to the brush 18 and dipping the brush 18 into the separate tubular container 22 to incorporate the second solution component C₂. In some such two-component tooth whitening solution embodiments, the solution component C₁ in the handle 16 includes a bleaching agent such as hydrogen peroxide or another peroxide-based bleaching agent, while the solution component C₂ in the container 22 includes a catalyst such as an enzyme, e.g. the enzyme papain, a pH buffer such as sodium hydroxide, or another chemically active component or mixture or combination of components that enhances decomposition of the bleaching agent or otherwise modifies the chemistry of the tooth whitening process. Alternatively, the bleaching agent may be stored in the tubular container 22 and the catalyst, buffer, or other additional component or components stored in the handle 16. In either arrangement, the decomposition of the peroxide-based bleaching agent is facilitated at the time of application by mixing with the catalyst or pH buffer.

The applicator 12 is merely an illustrative example—substantially any type of applicator suitable for paint-on application of a tooth whitening solution may be used. For example, the applicator could be a cotton-tipped or sponge-tipped swab, a square sponge sized to contact the teeth with some precision, a tubular applicator containing the tooth whitening agent and having a nozzle via which the tooth whitening agent is ejected responsive to squeezing the tube, a conventional toothbrush, or so forth. In some embodiments, two or more components of a multi-component tooth whitening solution are stored in separate reservoirs of a single tube, packet, or other single container, and are mixed or otherwise combined at the time of application by a twist mechanism or other mechanical mechanism that ruptures a barrier or barriers between the separate reservoirs to provide fluid communication therebetween. In another embodiment, a multi-component tooth whitening solution is contained in two or more separate applicators, one per component, in which each applicator includes its own brush, cotton swap tip, or so forth. The two or more components are then applied to the teeth in successive separate paint-on operations.

The paint-on tooth whitening solution, optionally after combining two or more components, is in some embodiments a relatively viscous fluid, such as a gel, that tends to produce a relatively uniform layer when painted onto a tooth using the applicator 12. Accordingly, the painted-on tooth whitening gel or solution forms a thin film of relatively controlled thickness so as to enable application of a precise dosage of the paint-on tooth whitening solution or gel C₁, C₂.

In one illustrative embodiment, the first component C₁ of the tooth whitening solution or gel is a mixture or solution of water, optional glycerine, hydrogen peroxide, ammonium hydroxide, phosphoric acid and a polyvinyl substance such as PVP. In this illustrative component, water provides functionality at least including acting as a solvent, the optional glycerine provides functionality at least including acting as a humectants, the hydrogen peroxide provides functionality at least including acting as a bleaching or whitening agent, ammonium hydroxide provides functionality at least including acting as a pH adjuster, phosphoric acid provides functionality at least including acting as a buffer, and PVP provides functionality at least including acting as a film former and stabilizer to promote uniform application as a film on the teeth. In some embodiments that include the optional glycerine, the percentage glycerine in the solution component is about 0-25%. The percentage of hydrogen peroxide is preferably greater than about 10%, and more preferably greater than about 12%, and may be 12%, 13%, 14%, 15%, 16%. 17%, 18%, 19%, 20%, 21%, 22%, 23%, 24%, 25%, 26%, or higher. Instead of hydrogen peroxide, another peroxide-based bleaching agent can be used such as percarbonate peroxide, urea peroxide, carbonide peroxide, or so forth. In such a case, the concentration is suitably selected to provide bleaching characteristics of the aforementioned hydrogen peroxide concentrations. For example, a given solution of percarbonate peroxide has bleaching characteristics similar to a solution of hydrogen peroxide of about one-third concentration. Thus, a solution of percarbonate peroxide is preferably greater than about 30% to provide bleaching characteristics similar to those of a hydrogen peroxide solution of concentration greater than about 10%. Furthermore, it is contemplated to employ a non-peroxide-based bleaching agent.

An optional second component C₂ in these illustrative two-component embodiments acts as a whitening activator. As an illustrative example, the second component C₂ may include purified water acting as the solvent, methyl paraben serving as a preservative, an optional catalyst promoting bleaching agent decomposition such as the enzyme papain, an optional thickening agent such as carbomer or a non-reactive thickening agent such as sodium carboxymethylcellulose, a sodium hydroxide pH buffer or adjusters optional glycerine, copolyvidonum, polysorbate 20, and an optional flavoring substance. Papain is believed to act as a stain scavenger and is also believed to promote decomposition of the peroxide-based bleaching agent so as to release oxygen free radicals, thus functioning as a catalyst to speed up the bleaching chemical reaction. Although papain is a preferred enzyme for catalyzing bleaching agent decomposition, other enzymes that catalyze bleaching agent decomposition can also be used. The optional glycerine acts as a humectant and copolyvidonum is a resin. Polysorbate acts as a surfactant/solubilizer. The optional flavoring agent, if included, is in some embodiments selected, and the article of manufacture labeled, in accordance with 21 C.F.R. §701.3(a).

While not being limited to any particular theory of operation, it is believed that the hydrogen peroxide or other peroxide-based bleaching agent releases oxygen free radicals that react with and cleave bonds to chromophores, providing a lightening or whitening effect in the color of the teeth. The one, two, or multi-component paint-on tooth whitening gel or solution is preferably configured to form a relatively thin film when painted onto the teeth using the applicator 12 so as to allow for precise control of the dosage of the high concentration hydrogen peroxide ingredient on the teeth.

In some embodiments, the tooth whitening solution or gel C₁, C₂ includes an oxygenating peroxide gel, such as a gel containing a peroxide-based bleaching agent, and an activator gel containing, for example, papain enzymes in a slightly higher pH range than the peroxide gel, thus enhancing the release of available oxygen from the bleaching agent through enzymatic action while buffering the pH of the gel on the surface of the tooth. The use of the papain enzymes enables rapid decomposition of the bleaching agent at a relatively neutral pH level. In contrast some existing two-component whitening systems rely on sodium hydroxide or another caustic soda compound as the primary catalyst for decomposing the bleaching agent, which results in a highly alkaline solution having a pH in the range of about 10.0-11.0. Without being limited to any particular theory of operation, it is believed that the more neutral operational pH achievable using the papain enzyme reduces the likelihood of inducing tooth sensitivity. Additionally, suspected causes of tooth sensitivity include tooth dehydration due to the use of glycerine as an anhydrous base. In some embodiments disclosed herein, an anhydrous base is not used, which is believed to further reduce a likelihood of induced tooth sensitivity. In some embodiments, no reactive thickener is included. Since a reactive thickener binds the peroxide and prevents its rapid disassociation, the absence of a reactive thickener such as carbomer accelerates liberation of oxygen from peroxide-based bleaching agents, which decreases the length of time of the tooth whitening procedure, which is also believed to reduce the likelihood of inducing tooth sensitivity and additionally increases a likelihood that the consumer will not prematurely remove the tooth whitening solution or gel.

The illustrated protective wrap or microtray assembly 14 includes a protective strip, wrap, microtray, or other conforming protective cover 24 which in the illustrated embodiment is a strip or wrap removably disposed on a backing paper 26 or other support. In other embodiments, the wrap may be wound into a roll, wound around a reel or other core, provided in unsupported strips, or otherwise packaged or configured. FIG. 1 shows the protective wrap 24 partially drawn off of the backing paper 26. Optionally, a coating 24s may be disposed on one side (as shown) or both sides of the protective wrap 24. The coating 24s can serve various purposes or combinations of purposes. In some embodiments, the coating 24s may comprise a substitute for the previously discussed second or other additional component C₂ of the tooth whitening solution. That is, in some embodiments the second or additional component C₂ is disposed with the protective wrap 24, for example being impregnated into the wrap 24 or included in the coating 24s, rather than being provided as a second paint-on component. For example, the optional whitening activator component of the tooth whitening solution including purified water, methyl paraben, papain, the non-reactive thickening agent, a pH buffer or adjuster, glycerine, copolyvidonum, polysorbate 20, and an optional flavoring substance may be formed as the coating 24s, or impregnated into the wrap 24, rather than being embodied as a second component of the paint-on tooth whitening solution. In such a case in which the coating 24s is chemically active and part of the tooth whitening chemistry, the coating 24s is optionally disposed on only the side of the wrap 24 that contacts the teeth, and when the protective wrap or microtray 24 is applied to the teeth after the paint-on operation the contacting coating 24s mixes with the painted on component to accelerate decomposition of the bleaching agent. In some embodiments, the coating 24s may include bleaching agent such as hydrogen peroxide. In some such embodiments, the bleaching agent concentration in the coating 24s is lower than the concentration in the painted-on tooth whitening solution or gel, and is intended to provide some limited whitening in areas the consumer may miss during the paint-on application. For example, in some embodiments, the coating 24s or the body of the protective wrap 24 may have a 0-7% hydrogen peroxide concentration while the paint-on tooth whitening solution or gel has a higher hydrogen peroxide concentration of around 10-25% or higher. In some embodiments, the coating 24s may serve other purposes, such as providing a breath freshening component, in which case the coating may be applied on both sides of the wrap or on the side that does not contact the teeth. In other embodiments, a breath freshening component may be embedded into the material of the wrap 24, rather than provided as a separate coating. Inclusion of ingredients on one or both sides of the wrap or other protective cover, or impregnated wholly or partway through the wrap or other protective cover, is also contemplated.

In some embodiments, the coating 24s may include an adhesive. In other embodiments, no adhesive is included, but rather the protective wrap or microtray 24 adheres to the teeth through the medium of the painted-on tooth whitening solution or gel. In these latter embodiments, the applied film of paint-on tooth whitening solution or gel has a stickiness effective to retain the conforming protective cover 24 conformally disposed over the teeth after the paint-on application of the tooth whitening solution or gel, and the protective wrap or microtray 24 is suitably pressed gently on the front of the teeth to effectuate sticking or bonding to the teeth via the medium of the painted-on solution or gel. The protective wrap or microtray 24 is in some approaches aligned approximately with the proximate gum line, and any excess wrap is suitably wrapped over the biting edge of the teeth. In other approaches, the wrap or microtray 24 is aligned with the bottom of the teeth (i.e., distal from the gumline) and is wrapped toward the gumline, with any excess wrap material extending over the gum line. In embodiments in which the protective wrap or microtray 24 does not contain a high concentration of bleaching agent, or contains no bleaching agent, it is typically not problematic if a portion of the protective wrap or microtray 24 extends over the gum line and contacts the gums or other soft tissue.

In some embodiments, the protective wrap or microtray 24 is made of a material that dissolves or breaks apart over time in the oral environment, i.e. in the mouth. For example, the protective wrap or microtray 24 may be made of a food grade cellulose material that dissolves in the mouth in a predetermined period of time. In embodiments in which the protective wrap 24 is chemically inert, that is, does not contain any chemicals that participate in the tooth whitening chemistry, the consumer may be advantageously informed of this so that the consumer is reassured about swallowing the dissolving wrap material.

The illustrated protective wrap or microtray 24 is readily applied and provides a close bond to the teeth. The edge of the wrap may be straight, or optionally an edge of the wrap may have some curvature to better conform with the gum line. The protective wrap or microtray 24 is configured to provide a conformal protective cover for the teeth after application of the paint-on whitening solution or gel. Although the illustrated wrap is convenient for the consumer, it is also contemplated to employ an otherwise configured conformal protective cover, such as a boil-and-bite tray, foam tray, a putty, gel, or other protective barrier layer that is painted on after the whitening composition C₁, C₂, or so forth. In the context of application in a dental office setting it is also contemplated to employ a custom-fitted tray as the conformal protective cover; however, it is expected that the illustrated protective wrap or microtray 24 will likely provide comparable results at substantially lower cost and less inconvenience compared with the construction and use of a custom-fitted tray.

Although not illustrated, the tooth whitening article of manufacture can include other components, such as a sheet, booklet, or other printed matter providing directions for use, an Internet URL printed on the protective wrap or microtray 24 or elsewhere providing a link to a website containing graphical directions for use such as a video or a series of illustrative slides or successive linked webpages, a web link or digital medium containing interactive audio or video for consultation purposes, a flash drive or other electrostatic memory device operable in conjunction with a computer (typically not included in the article of manufacture) to provide directions for use in multimedia format such as video or graphical slides, suitable packaging materials, or so forth. Moreover, it is contemplated for some article of manufacture embodiments to omit the applicator and provide only the paint-on tooth whitening solution or gel—in these article of manufacture embodiments, the consumer separately provides a suitable applicator, for example employing a conventional toothbrush or cotton swab or a sponge applicator.

The described tooth whitening components and article of manufacture embodiments are provided as illustrative examples. Other variations are also contemplated. For example, it is contemplated to include a photoinitiator that promotes decomposition of the bleaching agent responsive to light, or a selected spectrum of light, such a tooth whitener being used in conjunction with an actinic source of light such as a light emitting diode (LED), lamp, or the like. In some such embodiments, a phosphorescent component may be included in the tooth whitening gel or in the wrap 24 or the wrap coating 24s, or in one or more of the paint-on components C₁, C₂, which can be activated by exposure to light prior to being applied to the teeth. Alternatively, the actinic light source can be applied through the applied protective wrap if the wrap is translucent or transparent for the applied light, or can be applied to the protective wrap before its application if the wrap contains the light-sensitive component.

Certain illustrative tooth whitening solution compositions have been set forth herein merely as illustrative examples. Those skilled in the art can readily employ different compositions including more, fewer, or different components, different component percentages, and so forth. Advantages set forth herein of the combination of a paint-on whitener followed by application of a conforming or conformal protective cover are expected to be attained regardless of the particular whitening solution or combination of whitening solutions utilized. In general, the whitening solution has chemically active components that remove tooth stains, and these chemically active components can cause soft tissue irritation, overwhitening, induced tooth sensitivity, or other problems. By using the paint-on tooth whitener, placement of the generally irritating tooth whitening solution is well-controlled. By subsequently applying the conforming or conformal protective cover, the painted-on tooth whitener is held in place and soft tissue irritation is suppressed. Further, in some embodiments the conforming or conformal protective cover may include chemically active components such as additional bleaching agent, a catalyst enhancing bleaching agent decomposition, or so forth. Advantageously, by placing such components on the conforming or conformal protective cover their effect is initiated upon placement of the conforming or conformal protective cover onto the teeth.

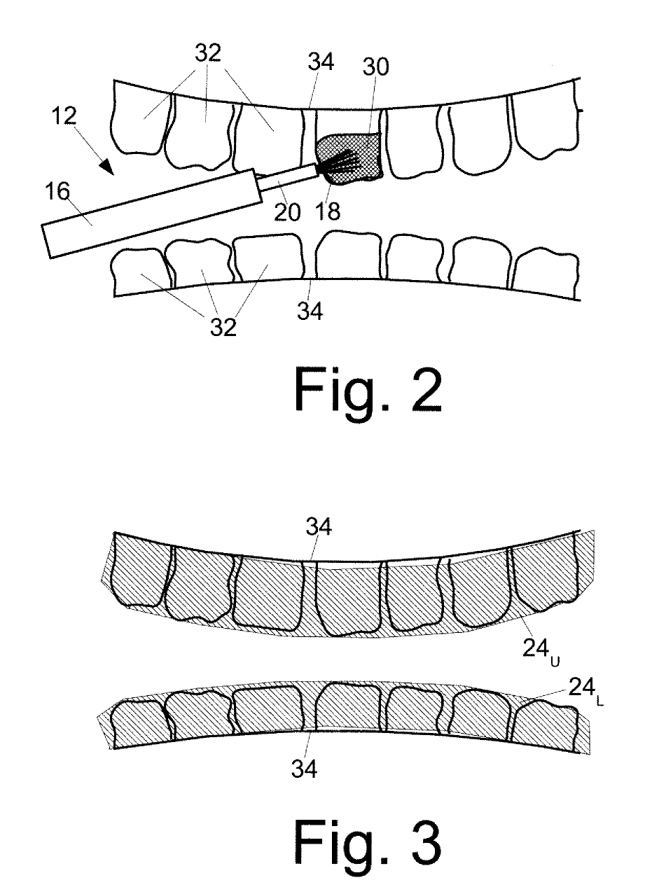

FIG. 2 shows the use of the applicator 12 to apply a film 30 of the paint-on tooth whitening solution or gel C₁ or C₁, C₂ (shown only in FIG. 1) on the outward facing surface of each tooth 32. In FIG. 2, the painting of one tooth is shown for illustrative purposes; however, each tooth that is desired to be whitened is preferably separately painted. Moreover, while FIG. 2 shows painting of a frontmost upper tooth first, some consumers may find it more convenient to paint the teeth in another order such as from left-to-right or from right-to-left, and/or may find it more convenient to start painting the lower teeth first followed by painting of the upper teeth. For a typical consumer, it is sufficient to paint the eight or so teeth closest to the front of the mouth as these are the teeth most commonly visible during ordinary conversation and other ordinary activities. However, more or fewer teeth can be painted. In painting on the applied film 30, one should try to avoid contact with the gum lines 34 or gum tissue.

In a suitable approach, an amount of the tooth whitening solution or gel C₁, C₂ is applied one tooth at a time to the upper and lower front eight teeth. If the tooth whitening solution or gel or component thereof C₁ is manually driven from a hollow interior of the handle 16 to the brush 18, then the handle components 16, 20 are suitably twisted, pumped, squeezed or otherwise manually operated occasionally to replenish the supply of the tooth whitening solution or gel on the brush 18. If the tooth whitening solution or gel or component thereof C₂ is contained in the separate container 22 (shown only in FIG. 1) then the brush 18 is occasionally dipped into the container 22 to replenish the supply of the tooth whitening solution or gel on the brush 18. In some embodiments, the applicator 12 is configured so that one twist, pump operation, or dip of the brush 18 into the container 22 provides a sufficient amount of tooth whitening solution or gel on the brush 18 to paint one tooth. In some cases, a consumer can paint the relevant front teeth in about one-half minute to one minute or faster using such an approach. More generally, the paint-on application time varies with the particular consumer. Because the tooth whitening solution or gel includes a relatively high concentration of bleaching agent, such as for example 10%, 12%, or higher concentration of hydrogen peroxide in some embodiments, it is advantageous to avoid having the tooth whitening solution or gel contact the gums. To avoid this, it is advantageous to brush the gel towards the biting edge, i.e. away from the gum line 34, so as to apply the thin film 30 of the tooth whitening solution or gel to the tooth.

If the tooth whitening solution or gel includes two components C₁, C₂ that are mixed prior to being painted on, or if the tooth whitening solution or gel has a single component (which could be either C₁ or C₂ in the illustrative applicator 12 shown in FIG. 1), then each tooth is painted once. On the other hand, if the tooth whitening solution or gel has two or more components that are not mixed together prior to paint-on application, then each tooth is painted two or more times, once using each component of the multi-component tooth whitening solution or gel. Different application elements (e.g., brushes, cotton swabs, or so forth) may be used for each component, or the same application element may be reused for two or more of the tooth whitening solution or gel components.

With reference to FIG. 3, after the paint-on tooth whitening solution or gel is applied as shown in FIG. 2, the consumer applies the protective wrap or microtray 24 over the painted teeth. In some embodiments, the painted on tooth whitening solution or gel is left exposed to air for a selected relatively short period of time, such as about 30 seconds in some embodiments, before applying the protective wrap or microtray 24. FIG. 3 shows the upper teeth covered by a first protective wrap or microtray 24_{U}, and the lower teeth covered by a second protective wrap or microtray 24_{L}. In a typical embodiment, both protective wraps or microtrays 24_{U}, 24_{L} are interchangeable and are suitably embodied as the protective wrap or microtray 24 shown in FIG. 1. Alternatively, it is also contemplated to have non-interchangeable wraps for the upper and lower teeth, for example to accommodate differences in the typical size and shape of the respective sets of upper and lower teeth. The protective wraps or microtrays 24_{U}, 24_{L} ensure that the ingredients of the painted-on tooth whitening solution or gel are held against the surface of the teeth without being removed by the lips, tongue or cheek.

The illustrated protective wraps or microtrays 24_{U}, 24_{L} are partially translucent such that the teeth are visible through the protective wraps or microtrays 24_{U}, 24_{L}. In other embodiments the protective wraps or microtrays 24_{U}, 24_{L} may be wholly opaque such that the teeth are not visible, or semi-opaque. In embodiments in which the protective wraps or microtrays 24_{U}, 24_{L} do not include a high concentration of bleaching agent, it is generally not problematic for the protective wraps or microtrays 24_{U}, 24_{L} to contact the gum lines 34. If the protective wraps or microtrays 24_{U}, 24_{L} include a coating 24s on one side that is active in the tooth whitening chemistry, then this coating 24s should face and contact the teeth. The exposed surface of the applied protective wraps or microtrays 24_{U}, 24_{L} is typically uncoated or optionally coated with a flavoring agent, breath freshener, or the like. If a coating 24s is provided and is not readily visible, the protective wraps or microtrays 24_{U}, 24_{L} optionally include an indication of which side to apply to the teeth, for example as text or symbols printed on the protective wraps or microtrays 24_{U}, 24_{L}. On the other hand, if no coating is provided then the protective cover wrap may advantageously have interchangeable sides, either of which can be pressed against the painted teeth.

Once applied, the protective wraps or microtrays 24_{U}, 24_{L} are left in place during the whitening process, for example about 10-15 minutes in some embodiments, although longer or shorter whitening times are also contemplated. The high concentration of bleaching agent in the painted on tooth whitening solution or gel enables such a short processing time. Additionally, the optional absence of a reactive thickening agent and/or the optional inclusion of a catalyst or enzyme such as papain further shorten the processing time. Once the tooth whitening process is completed, the protective wraps or microtrays 24_{U}, 24_{L} are removed. In some embodiments, the removal is automatic because in these embodiments the protective wraps or microtrays 24_{U}, 24_{L} are made of a material that degrades in the mouth, such as food grade cellulose material. The thickness, density, or other properties or combination of properties of such dissolvable wraps or microtrays are selected to ensure that the protective wraps or microtrays 24_{U}, 24_{L} will remain in place long enough to provide protection during the tooth whitening process before dissolving or breaking apart. In some embodiments, the protective wraps or microtrays 24_{U}, 24_{L} are not dissolvable, and instead the consumer removes the protective wraps or microtrays 24_{U}, 24_{L} after use. In some such embodiments, the protective wraps or microtrays 24_{U}, 24_{L} may include a visual change agent that changes the color or other visually perceptible characteristic of the protective wraps or microtrays 24_{U}, 24_{L}, such as causing the wrap to turn transparent or translucent, after a time interval of exposure to the saliva another element of the oral environment. The time interval of exposure for the color change is selected to be as long as or longer than the time interval over which the tooth whitening process proceeds to completion. In this way, the protective wraps or microtrays 24_{U}, 24_{L} are configured to change color, or turn transparent, or turn opaque, or provide another visual indicia, to alert the consumer that the protective wraps or microtrays 24_{U}, 24_{L} may now be removed. The strip may also be impregnated with a dye that changes color over a prescribed period of time and visually alerts the consumer when that prescribed period of time has elapsed where the consumer can remove the strip. In some embodiments, the color change is contemplated to be configured to occur over different time intervals to help consumers whiten stained teeth that are typically more difficult to remove in a shorter period of time. For example, if the consumer has naturally yellow teeth or lightly stained teeth, the color might change from its original shade to clear after a few minutes or less of wear. On the other hand, if the consumer is a heavy coffee drinker, an FD&C dye or other coloring agent that might be used to give color to the strip might be of more intense chroma or using a coloring agent that would slowly change to or from a clear color over a longer period of time. Nicotine stained teeth would require a strip that would change color over a longer period of time. In some embodiments, the choice of color change rate or time interval is correlated with the bleaching time, so that a longer bleaching time is associated with a protective strip or microtray that changes color more slowly or after a longer interval.

The described process including the paint-on operation described with reference to FIG. 2 and the wrapping operation described with reference to FIG. 3 together define a single tooth whitening session. The session typically takes 30 minutes or less, and more preferably takes about 10-15 minutes, and more preferably between 30 seconds and 10 minutes. Longer or shorter session times are also contemplated. The protective wrap or microtray 24 can be removed after use. The removal can be manual in the case of a non-dissolving wrap or microtray, or the removal can be automatic through dissolution in the case of a dissolving wrap or microtray. The tooth whitening session is optionally repeated, for example once or twice a day, as part of an ongoing tooth whitening regimen to obtain and maintain whitened teeth. In some embodiments, perceptible tooth whitening is obtained in a single session, with further tooth whitening achieved by repeated sessions. In a typical embodiment using about 10-25% hydrogen peroxide in the paint-on solution and the disclosed optional papain enzyme and optionally a non-reactive thickener, substantially complete tooth whitening is typically achieved in about 7-14 days, although other time frames may be observed depending upon the extent and type of initial discoloration of the teeth, the precise whitening solution or gel composition, and other factors. Continued maintenance of the tooth whitening regimen after this period advantageously maintains white teeth. Such proactive use allows for protection against future stains as the whitening ingredients generally remove stains faster than the time it takes for new stains to absorb into the teeth.

The combination disclosed herein of a paint-on whitening solution or gel applied using the applicator 12 followed by application of the protective strip or microtray 24 has the apparent disadvantage of higher cost versus a single-step approach (i.e., either a paint-on whitener used alone, or a strip-type whitener impregnated with bleaching agent used alone), and the further apparent disadvantage that the consumer is called upon to perform two separate operations, whereas existing paint-on whiteners or existing strip-type whiteners each entail only a single operation. However, the inventor has recognized that the combination of applying a paint on whitening solution or gel followed by application of the protective strip or microtray 24 provides numerous advantages which are not readily apparent, and which collectively are believed to outweigh the inconvenience and additional cost of employing a two-step process. A non-limiting exposition of some of these synergistic advantages of combining a paint-on whitener with a subsequently applied protective strip or microtray is now set forth.

By using a paint-on whitening solution or gel, a precise placement of whitening solution is achieved. As a thin film, the layer 30 of paint-on whitening solution or gel provides a precise placement of the whitening solution or gel. The combination of paint-on whitener followed by application of the protective cover facilitates use of a higher concentration of bleaching agent without concomitant soft tissue irritation. In contrast, a whitening strip impregnated with whitening solution may be more prone to producing gum irritation, enhanced tooth sensitivity, or other problems. With reasonable care the paint-on whitening solution can be applied to the teeth using the illustrated applicator 12 or another suitable applicator without substantial deposition of the whitening solution onto the gums.

By subsequently applying the protective strip or microtray 24, the likelihood of transfer of the whitening solution to the lips, gums, or other proximate soft tissue due to oral activity such as talking, breathing, or so forth is substantially reduced. This enables the concentration of bleaching agent in the paint-on whitening solution or gel to be substantially increased. In some embodiments, it is contemplated for the concentration of bleaching agent in the paint-on whitening solution or gel to be the equivalent of about 10% hydrogen peroxide or higher. Because of the precise dosage delivered by thin-film paint-on application and the protection of soft oral tissue provided by the protective strip or microtray 24, this high concentration of bleaching agent does not lead to overwhitening, gum irritation, enhanced tooth sensitivity, or other problems for most consumers.

Moreover, in some embodiments the reactive thickener is replaced by a nonreactive thickener such as sodium carboxymethylcellulose. This has the effect of further increasing the speed of the bleaching process as compared with processes employing a reactive thickener such as Carbopol®. As used herein, a non-reactive thickener is one which does not substantially react with the bleaching agent or slow down generation of oxygen free radicals from the bleaching agent.

The protective strip or microtray 24 is optionally chemically inert, that is, includes no chemical components designed to enhance the whitening process. In this case, there is no call for special handling of the protective strip or microtray 24, in contrast to chemically active whitening strips that contain hydrogen peroxide and typically other chemicals. Moreover, a chemically inert strip has the advantage that it can be configured to be applied with either side facing the teeth.

In other embodiments, the protective strip or microtray 24 includes selected chemically active components, for example in the form of the coating 24s, that contribute to the whitening process. For example, in some embodiments the protective strip or microtray 24 includes a catalyst such as an enzyme that promotes the decomposition of the bleaching agent and consequent release of oxygen free radicals. In some embodiments, the enzyme papain is suitably used, although other enzymes or other catalysts are also contemplated. Such enzymes or catalysts are selected to react with and accelerate decomposition of the peroxide-based bleaching agent to expedite the release of oxygen free radicals, thus speeding up the bleaching process so as to further reduce the duration of the whitening session.

If the catalyst or other decomposition accelerator is incorporated into the protective strip or microtray rather than into the painted-on whitening solution, then it is not combined with the painted-on whitening solution until the protective strip or microtray is applied. As a result, the enhanced whitening provided by the decomposition accelerator is controlled in both time and area of application. In other embodiments, the protective strip or microtray includes other chemically active components such as sodium hydroxide or another pH buffering agent, so as to maintain a pH level conducive to bleaching agent decomposition at an enhanced rate. In other embodiments, the catalyst or other contributory component is added to the paint-on tooth whitening solution at the time of application, for example by providing two vials or tubes whose contents are combined shortly before the paint-on operation is initiated.

The combination of higher bleaching agent concentration, optional reduced or eliminated buffering through the use of a nonreactive thickener, and optional catalyzing or other bleaching agent decomposition-accelerating chemicals on the protective strip or microtray, ensures a fast whitening process. In some embodiments disclosed herein, a whitening session can be performed in its entirety in about 2-15 minutes, after which the protective wrap, strip, or tray 24 can be removed by dissolving or manual removal.

Still further, the disclosed tooth whitening procedures enable new paradigms for initiating or guiding a tooth whitening regimen. Existing tooth whitening procedures are designed to be performed by a dentist, or by the consumer. In the former case, the invasive nature of the dental procedure entailing insertion of a lip spreader, precise application of a gum sealant, or so forth tends to restrict such tooth whitening regimens to being performed by a professional dentist in a dental office or similar setting. Indeed, in the United States and some other countries, dental regulations require that such procedures be performed only by licensed professional dentists. On the other hand, existing over-the-counter tooth whitening procedures entail long application times due to the typically low concentration of bleaching agent. Because of these long tooth whitening session durations, each session is typically performed at night or early morning, in the privacy of the consumer's home and in the presence of a washroom sink or the like. Such long procedures are not conveniently performed in a guided fashion.

The disclosed procedures, however, are well-suited for guided initiation. The objective of such initial guidance is to train the consumer during the initial one, two, or few whitening sessions, after which the subject is well prepared to engage in subsequent tooth whitening sessions unassisted. Another objective of such initial guidance is to provide the consumer with quantitative feedback as to the effectiveness of the tooth whitening session.

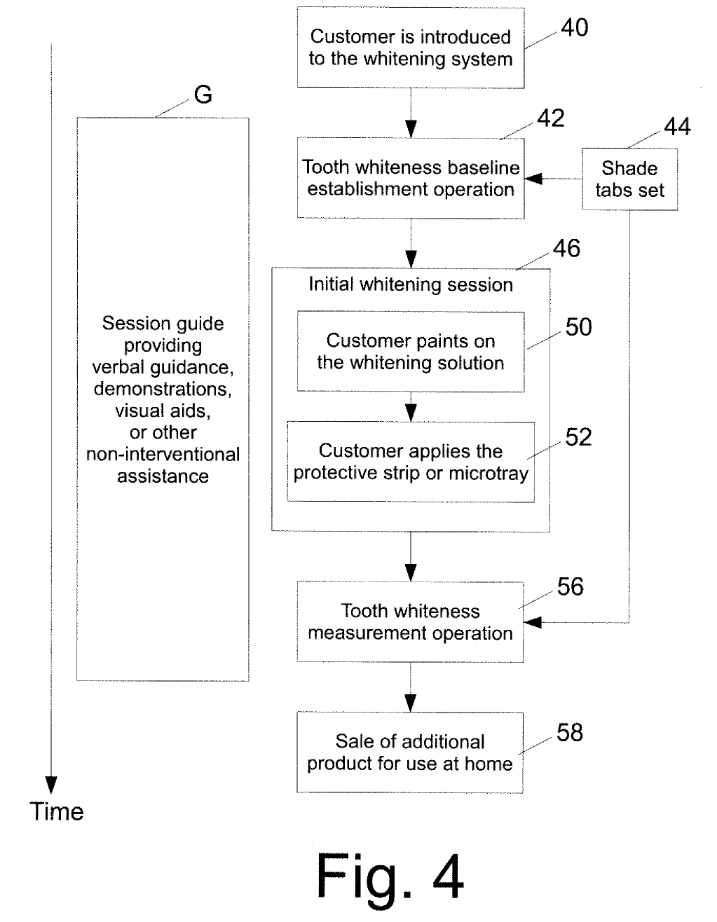

With reference to FIG. 4, a suitable guided initiation is described. In an operation 40, the consumer is introduced to the tooth whitening system. This can occur in various ways. For example, the consumer may be given a free initial tooth whitening session with the option to buy the product for use in future guided or unguided whitening sessions as part of a tooth whitening regimen if the consumer is satisfied with the results obtained in the free initial session. In another approach, the initial guided session is free but employs product that was purchased by the consumer. In yet other approaches, the consumer may be charged for both the product and the guidance provided in the initial guided session.

The initial guided session is advantageously guided by a human guide, either in-person or via a two-way interactive audio-video conference employing webcams or the like. The guide provides guidance G such as verbal guidance, demonstrations, possibly employing visual aids such as charts, photographs, or so forth, and other non-interventional assistance. The guidance G is preferably non-interventional, that is, does not involve the guide contacting the consumer's lips, mouth, or teeth either directly or indirectly (e.g., using dental instruments or gloves). The non-interventional guidance G typically includes advising the consumer as to how to perform the procedure, and providing quantitative feedback as to the effectiveness of the guided tooth whitening procedure. This correlates with the objective of the initial guided session, which is to train the consumer to perform subsequent tooth whitening sessions alone and without assistance or guidance.

In a baseline establishment operation 42, the consumer's tooth whiteness is measured. In some embodiments the session guide, who may, or may not, be a licensed professional dentist, utilizes a set of shade tabs 44 made of porcelain or another material to perform the whiteness measurement. The session guide holds up selected shade tabs for comparison with the consumer's teeth to find the closest matching whiteness. The shade tabs 44 generally provide more accurate measurement of whiteness than other measurement instruments such as a whiteness sheet that includes a line or array of patches representative of different tooth whiteness levels. However, it is also contemplated for the measurement operation 42 to employ a whiteness sheet or other measurement device. In some embodiments, the session guide may use a mirror to enable the consumer to personally compare the baseline tooth whiteness with the various shade tabs so that the consumer personally approves the baseline tooth whiteness. In the case of a session in which the guide is virtually present via an audio/video connection, the guide may ask the consumer to hold each test shade tab up to the consumer's teeth while looking into the camera, so as to enable the remote guide to view the comparison. The closest shade tab is preferably noted by being set aside, identified on a sheet of paper, or so forth, so as to provide the baseline whiteness value for comparison with the consumer's teeth after completion of the whitening session.

With the whiteness baseline established, the consumer performs an initial whitening session 46 with the advice and guidance of the session guide. The initial whitening session 46, like each subsequent whitening session, entails two successive operations. First, the consumer paints on the tooth whitening solution or gel C₁, C₂ using the applicator 12 in a paint-on operation 50 already described with reference to FIGS. 1 and 2. Second, the consumer applies the protective strips or microtrays 24_{U}, 24_{L} as already described with reference to FIGS. 1 and 3 in an application operation 52. During the application 50 of the paint-on whitening solution, the session guide observes and provides guidance G. For example, the session guide may initially explain to the consumer how to operate the applicator 12, for example showing the consumer how to draw out a portion of the whitening solution or gel C₁ by twisting or otherwise appropriately manipulating the applicator 12. The session guide may, as another example, then observe that the consumer is applying the paint-on solution or gel across or inadvisably close to the gum line 34, and accordingly instruct the consumer to avoid contacting gum tissue with the whitening solution. The session guide may explain, possibly with reference to an instructional video, demonstration model of the teeth, graphical cartoon, or so forth, that the consumer should preferably apply the paint-on whitener starting with the brush 18 close to the gum line 34 and brushing away from the gum line 34 toward the biting edge of the tooth, so as to minimize the likelihood of inadvertently getting whitener solution or gel onto the gums. The session guide may also provide guidance as to how much of the paint-on whitening solution or gel to apply, in order to achieve the desired thin film coating 30. During the application 52 of the protective wrap or microtray 24, the session guide may again provide guidance G as to the proper manner in which to apply the protective wrap or microtray 24. For example, the guidance G may include instructions to line up the edge of the microtray 24 with the biting edge of the teeth, gently pressing the microtray over the teeth and wrapping any excess onto the gum tissue. Alternatively, the guidance G may include the instructions to avoid contact with the gum tissue and start by placing the leading edge of the microtray 24 close to the cervical portion of the teeth, gently pressing the microtray 24 on the teeth and wrapping any excess over the biting edge of the teeth. For a consumer with short teeth, the guidance G may include instructions to use scissors prior to placing the microtray 24 onto the teeth so as to cut the microtray 24 to decrease its width to better fit the consumer's short teeth. In the case of a dissolvable wrap, the session guide may provide guidance G such as recommending that the consumer salivate and swallow occasionally to promote decomposition and removal of the dissolvable protective wrap or microtray 24 and to suppress the inadvertent pooling of excessive saliva.

It will be recognized that the operations 50, 52 are readily mastered and subsequently performed by most consumers without further assistance once the consumer learns how to do so properly in the guided initial whitening session based on the guidance G provided by the session guide. However, without such initial guidance G, many consumers may err in the initial application and obtain unsatisfactory results that deter the consumer from continued use of the product. Moreover, without such initial guidance G including the use of shade tabs 44, some consumers cannot accurately determine whether any tooth whitening has occurred responsive to a single session.

Because the objective is to teach the consumer to perform subsequent tooth whitening sessions unassisted, the session guide typically does not physically assist in performing the whitening session, and indeed in some embodiments does not physically contact the consumer's lips, mouth, or teeth. This is inherent in embodiments in which the session guide is only virtually present via a two-way audio-video conference. Additionally, whether present in-person or remotely, as long as the session guide does not contact the consumer's lips, mouth, or teeth either directly or via a dental instrument, it follows that the guided tooth whitening procedure is manifestly not an interventional dental procedure. This is an advantage in the United States and in certain other countries or jurisdictions in which interventional dental procedures can only be legally performed by a licensed dental professional. In the guided whitening procedures disclosed herein such as the illustrative initial guided whitening session shown in FIG. 4, the session guide does not perform an interventional dental procedure, and accordingly the session guide providing the guidance G can be someone other than a licensed professional dentist, although it is also contemplated for the session guide to be a licensed professional dentist.

As a consequence, the various guided whitening sessions disclosed herein including the illustrative guidance G can be performed in a dental office, but also can be performed in substantially any other reasonably sanitary setting. For example, the various guided whitening sessions disclosed herein can be performed in a beauty salon with a hairdresser or other salon employee serving as the session guide. Similarly, the various guided whitening sessions described herein can be performed in department stores, drug stores, mall kiosks, spas, airports, tanning salons, nail salons, optical centers, and so forth by employees or other personnel who are optionally not licensed professional dentists.

In an operation 56, a second whiteness measurement is performed. Because a session of the whitening techniques disclosed herein can be performed in a relatively short period of time, it is feasible for the session guide and the consumer to wait until the guided session is complete in order to perform the second whiteness measurement 56. The second whiteness measurement 56 is performed similarly and comparably to the baseline whiteness measurement 42, and the results are therefore properly comparable. In this way, the consumer and the session guide can quantitatively assess the effectiveness of the whitening procedure 46. Because the whitening techniques disclosed herein employ high bleaching agent concentration and are generally rapidly effective for most consumers, the second whiteness measurement 56 will typically show measurable improvement versus the baseline whiteness measurement 42. If a mirror is provided, the consumer can see the improvement directly, by comparing the teeth after the whitening session 42 with the shade tab selected in the baseline measurement 42 as representative of the whiteness of the consumer's teeth prior to the whitening session 46. This provides the consumer with quantitative feedback on how well the whitening procedure works, and enables the consumer to make an informed decision as to whether or not to continue the whitening treatment, which typically entails a purchase 58 of additional tooth whitening product, such as purchase of the tooth whitening article of manufacture illustrated in FIG. 1, for home use. In some embodiments, the consumer may be given one or more free sample tooth whitening articles of manufacture to enable the consumer to continue the tooth whitening regimen at home for one or a few more sessions before optionally making the purchase 58. Subsequent tooth whitening sessions can be either guided or unguided, although the goal is typically for the consumer to be able to perform future whitening sessions unguided after one, two, or a few guided sessions such as the illustrative guided session described with reference to FIG. 4.

In some instances, the improvement indicated by comparison of the second whiteness measurement 56 with the baseline whiteness measurement 42 may be less than satisfactory. In some such instances, the session guide may recommend repeating the procedure using a different whitening formulation. For example, if the consumer is a heavy smoker, the session guide may suggest trying a different whitening formulation that includes the bleaching agent and also a component designed to attack nicotine stains. Or, the session guide may suggest trying a different whitening formulation that includes a higher concentration of the bleaching agent. In such a case, the whitening operation 46 is repeated using the different whitening formulation, and a repetition of the second whiteness measurement 56 (i.e., a third whiteness measurement) is then used to quantitatively assess the effectiveness of the different whitening formulation.

In some embodiments, the consumer may return on occasion for a refresher guided whitening session, to ensure that the consumer continues to properly use the whitening system. In some embodiments, the session guide during such a refresher session may reevaluate the whitening formulation that the consumer is using. For example, after the consumer has been following the tooth whitening regimen for a while, the session guide may recommend in a refresher guided session that a whitening formulation with a lower concentration of bleaching agent is sufficient to maintain suitably white teeth for that particular consumer. Contrastingly, the session guide may recommend a whitening formulation with a higher concentration due to a change in the consumer's habits that is leading to enhanced tooth staining, such as taking up smoking or coffee drinking.

The tooth whitening methods and apparatuses disclosed herein have been described with reference to a “consumer” which is reflective of a typical arrangement in which the tooth whitening article of manufacture, such as that shown in FIG. 1, is sold to a consumer who the uses the tooth whitening article of manufacture in an ongoing tooth whitening regimen. However, it is to be understood that the consumer may be a paying consumer, a non-paying client consumers a hospital patient, an eligible health club member, or substantially any other person who may utilize (that is, consume) the tooth whitening methods and apparatuses disclosed herein.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. (canceled)

2. The tooth whitening method as set forth in claim 3, wherein the tooth whitening solution or gel includes a peroxide-based bleaching agent at a hydrogen peroxide-equivalent concentration greater than or about 18 percent.

3. A tooth whitening method comprising:
painting a tooth whitening solution or gel including a bleaching agent onto teeth, the tooth whitening solution or gel including a peroxide-based bleaching agent at a hydrogen peroxide-equivalent concentration greater than or about 15 percent; and
subsequent to the painting, disposing a conforming protective cover over the teeth.

4. (canceled)

5. A tooth whitening method comprising:
painting a tooth whitening solution or gel including a bleaching agent onto teeth, the tooth whitening solution or gel including a peroxide-based bleaching agent at a hydrogen peroxide-equivalent concentration greater than or about 14 percent, the tooth whitening solution or gel further including a non-reactive thickening agent that is substantially non-reactive with the peroxide-based bleaching agent; and
subsequent to the painting, disposing a conforming protective cover over the teeth.

6. The tooth whitening method as set forth in claim 5, wherein the tooth whitening solution or gel further includes a bleaching agent decomposition catalyst that accelerates decomposition of the peroxide-based bleaching agent.

7. The tooth whitening method as set forth in claim 3, wherein the painting and disposing are performed by the consumer in a non-dental office setting.

8. The tooth whitening method as set forth in claim 3, wherein the painting and disposing are performed under the non-interventional guidance of a session guide at a location selected from the group consisting of: a beauty salon, a department store, a drug store, a mall kiosk, a spa, an airport, a tanning salon, a nail salon, and an optical center.

9. The tooth whitening method as set forth in claim 3, wherein the conforming protective cover is configured to dissolve or break apart in the oral environment in about 2-15 minutes.

10. The tooth whitening method as set forth in claim 3, wherein the conforming protective cover is configured to change its visual appearance after about a preselected time in the oral environment.

11. The tooth whitening method as set forth in claim 3, wherein the conforming protective cover is a protective strip, wrap, or microtray, and the disposing comprises wrapping the conforming protective strip, wrap, or microtray over the teeth to ensure that the painted-on tooth whitening solution or gel is held against the surface of the teeth without being removed by the lips, tongue or cheek.

12. The tooth whitening method as set forth in claim 3, wherein the conforming protective cover is chemically inert.

13. The tooth whitening method as set forth in claim 3, wherein the conforming protective cover includes a catalyst configured to accelerate decomposition of the bleaching agent and consequent release of oxygen free radicals.

14. (canceled)

15. The tooth whitening article of manufacture as set forth in claim 16, wherein the conforming protective cover is not by itself effective to provide tooth whitening.

16. A tooth whitening article of manufacture comprising:
a tooth whitening solution or gel including a bleaching agent, the tooth whitening solution or gel configured for paint-on application onto teeth; and
a conforming protective cover configured to be conformally disposed over the teeth after painting with the tooth whitening solution or gel, wherein the conforming protective cover is chemically active to accelerate release of oxygen free radicals from the bleaching agent.

17. A tooth whitening article of manufacture comprising:
a tooth whitening solution or gel including a bleaching agent, the tooth whitening solution or gel configured for paint-on application onto teeth, the whitening solution or gel including a peroxide-based bleaching agent at a hydrogen peroxide-equivalent concentration of greater than or about 16 percent; and
a conforming protective cover configured to be conformally disposed over the teeth after painting with the tooth whitening solution or gel.

18. The tooth whitening article of manufacture as set forth in claim 17, wherein the whitening solution or gel includes a peroxide-based bleaching agent at a hydrogen peroxide-equivalent concentration of greater than or about 20 percent.

19. The tooth whitening article of manufacture as set forth in claim 16, wherein the whitening solution or gel includes a peroxide-based bleaching agent at a hydrogen peroxide-equivalent concentration of greater than or about 14 percent.

20. The tooth whitening article of manufacture as set forth in claim 17, wherein the whitening solution or gel further includes a catalyst that accelerates decomposition of the peroxide-based bleaching agent.

21. The tooth whitening article of manufacture as set forth in claim 17, wherein the whitening solution or gel does not include a reactive thickening agent.

22. (canceled)

23. The tooth whitening article of manufacture as set forth in claim 17, further comprising:
a paint-on applicator including an application element.

24. The tooth whitening article of manufacture as set forth in claim 23, wherein the paint-on applicator further includes a container, the tooth whitening solution or gel including at least one component disposed in the container and accessed using the application element.

25. (canceled)

26. The tooth whitening article of manufacture as set forth in claim 17, wherein the conforming protective cover is a protective strip, wrap, or microtray that is wrapped over the teeth to ensure that the painted-on tooth whitening solution or gel is held against the surface of the teeth without being removed by the lips, tongue or cheek.
